(19) United States
(12) Patent Application Publication
(10) Pub. No.: US 2024/0065528 A1
(43) Pub. Date: Feb. 29, 2024

(54) ENDOSCOPE HAVING A COMBINED VALVE HOUSING

(71) Applicant: AMBU A/S, Ballerup (DK)

(72) Inventors: Tim CLOSS, Kissing (DE); Martin Johst CHRISTENSEN, Copenhagen 0 (DK)

(73) Assignee: AMBU A/S, Ballerup (DK)

(21) Appl. No.: 18/270,259

(22) PCT Filed: Jan. 11, 2022
(86) PCT No.: PCT/EP2022/050399

§ 371 Date: Jun. 29, 2023

(30) Foreign Application Priority Data
Jan. 12, 2021 (DE) 10 2021 100 386.5

(51) Int. Cl. A61B 1/00; A61B 1/015

(52) U.S. Cl. CPC A61B 1/00068; A61B 1/00103; A61B 1/015; A61B 1/00045

(57) ABSTRACT

An endoscope including a handle including an aperture and a valve insert arranged in the aperture; the valve insert including: a first valve cylinder having a first cylinder wall, a second valve cylinder having a second cylinder wall, and a flange connecting and spacing apart the first valve cylinder and the second valve cylinder. The flange is arranged in the aperture of the handle. Portions of the first valve cylinder and of the second valve cylinder are arranged inside the handle and include a plurality of tube ports.

The present disclosure relates to an endoscope having a combined housing for valves. In particular, the present disclosure provides an endoscope comprising: an endoscope handle having an aperture; and a valve insert or valve housing arranged in said aperture. The valve insert is configured to form a valve housing for at least two valves.

### PRIOR ART

Endoscopes and similar specialized instruments such as bronchoscopes, arthroscopes, colonoscopes, gastroscopes, laparoscopes and duodenoscopes are well known from the state of the art and are used for visual examination and diagnosis of hollow organs and body cavities, as well as to assist in surgery, e.g. for a targeted tissue sampling. Basically, a distal tip unit of an endoscope, which is connected to an endoscope handle via a bending section and an insertion tube, can be inserted into a hollow organ or body cavity to be investigated with the endoscope. Both reusable and disposable endoscopes are known from the state of the art.

From the prior art, it is basically known to provide both a suction valve and a gas/water injection valve in the endoscope handle.

The suction valve usually has two tube ports, namely a first tube port connected to a working channel of the endoscope (preferably via a suction tube and a biopsy connector having a biopsy valve) and a second tube port connected to a suction device like a vacuum pump (via another suction tube). The suction valve is basically configured to control a suction through the working channel, i.e. a suction that shall be applied to an object to be examined (e.g. hollow organ or body cavity). In particular, the suction valve may be brought into a valve closed state (in which no suction is applied to said object) and a valve opened state (in which suction is applied to said object).

The gas/water injection valve usually has a plurality of tube ports. In particular, it is known to connect tube ports of the gas/water injection valve to suitable gas/air/water sources (e.g. water tank or air pressure/carbon dioxide source). Moreover, it is known to connect tube ports with tubes provided in and extending along an insertion cord (comprising the insertion tube, the bending section and the distal tip unit) of the endoscope. An operator, in particular physician, can bring the gas/water injection valve into different positions/states, in order to reach different purposes.

E.g., the gas/water injection valve may be controlled such that a positive air pressure source provides positive air pressure to an insufflation tube provided in the insertion cord. In this way, positive air pressure may be provided in order to inflate an organ like the intestine or the stomach. It is to be understood that gas used for the insufflation through the gas/water injection valve is most often CO2 (carbondixode). Inflating an organ may help to observe a surgical intervention and may facilitate a further insertion of the endoscope. In addition, the gas/water injection valve may also be controlled such that a water tank provides water under pressure to a rinsing tube provided in the insertion cord. Providing water under pressure may e.g. help to rinse a soiled optical system in the distal tip unit.

Usually the suction valve and the gas/water injection valve are two separate valves. A prior art endoscope is e.g. known in which the suction valve is inserted into a first aperture provided in the endoscope handle and in which the gas/water injection valve is inserted into a second aperture provided in the endoscope handle. Both, the suction valve and the gas/water injection valve comprise a plurality of tube ports. In each tube port of the plurality of tube ports a sealing ring is usually inserted in order to provide a fluid-tight connection between the respective tube port and a tube to be fixed to said tube port. Typically, the tube is fixed to the tube port by gluing.

As is easily understood, the above described prior art endoscope has disadvantages with respect to its manufacturing and assembly. In particular, the suction valve and the gas/water injection valve have to be manufactured as entirely separate parts. Moreover, both the suction valve and the gas/water injection valve have to be separately assembled into the endoscope handle. In addition, sealing rings have to be inserted into each tube port, and tubes have to be separately glued to each tube port.

From the prior art, in particular document U.S. Pat. No. 6,383,132 B1, it is also already known to provide an endoscope comprising: an endoscope handle having an aperture; and a valve insert arranged in said aperture, wherein the valve insert forms a valve housing for two valves. U.S. Pat. No. 6,383,132 B1 discloses a valve module having basically a cuboid shape. The valve module is positioned in a recess provided at a proximal end of a housing of the endoscope handle. The valve module comprises sealing points, which are provided to sealingly connect the valve module to channels/conduits leading to external air/water sources or leading towards the distal tip unit.

The endoscope disclosed in U.S. Pat. No. 6,383,132 B1 has the disadvantage that a quality test of the valve module outside the endoscope is basically not possible, as the sealing points are not suited to easily attach a tube thereto. It is to be understood that it is basically desirable to conduct a quality test outside the endoscope and to only assemble the valve module into the endoscope handle in case the quality test has been passed. Moreover, the valve module disclosed in U.S. Pat. No. 6,383,132 B1—due to its cuboid shape—is a heavy metal part, which is disadvantageous with respect to handling and costs. In particular, in single-use-endoscopes, it is undesirable to provide a valve insert or valve module made from a solid metal material. The valve module disclosed in U.S. Pat. No. 6,383,132 B1 cannot be easily produced using a polymer material.

### BRIEF DESCRIPTION OF THE DISCLOSURE

The tasks and objectives of the present disclosure are to eliminate or at least to reduce the disadvantages of the prior art. In particular, a (single-use) endoscope having valves shall be provided which can be easily manufactured and assembled, and in which the valves can be simply quality tested outside the endoscope.

The tasks and objectives are solved by an endoscope in accordance with claim 1 and by a system in accordance with claim 15. Advantageous embodiments are claimed in the dependent claims and/or are explained below.

The present disclosure relates to an endoscope, in particular single-use-endoscope, comprising: an endoscope handle comprising an aperture; and a valve insert/housing/unit arranged in the aperture. The valve insert comprises: a first valve cylinder having a first cylinder wall, a second valve cylinder having a second cylinder wall, and a flange connecting and spacing apart the first valve cylinder and the second valve cylinder. The flange is arranged in the aperture of the endoscope handle, and portions of the first valve cylinder and of the second valve cylinder are arranged inside the endoscope handle and comprise a plurality of tube ports.

Said differently, the present disclosure provides an endoscope having a combined housing for valves. In particular, the valve insert of the present disclosure is configured to form a valve housing for at least two valves. The valve insert comprises two valve cylinders (the first valve cylinder and the second valve cylinder) which both have a cylindrical shape and are formed as hollow cylinders. Each of the two hollow valve cylinders is thus formed by a cylinder wall having a specific, in particular constant, wall thickness. In particular, the first valve cylinder is formed by the first cylinder wall, and the second valve cylinder is formed by the second cylinder wall. A flange is provided which connects and spaces apart the two valve cylinders. This flange is intended to be arranged in and fixed to the aperture of the endoscope handle. When the flange is fixed to the aperture, both the portion of the first valve cylinder, which comprises tube ports, and the portion of the second valve cylinder, which comprises tube ports, are arranged inside the endoscope handle, so that tube connections are not visible from the outside in the assembled state of the endoscope.

The present disclosure enables that only one part (the valve insert) has to be manufactured, which serves as a valve housing for two valves. It is thus not necessary anymore to manufacture two housings for the two valves. This also means that only one part (the valve insert) has to be assembled to the endoscope handle. As the first valve cylinder and the second valve cylinder have a cylindrical shape and are formed as hollow valve cylinders having a rather small wall thickness, and as the two valve cylinders are connected by a thin-walled flange, it is possible to easily manufacture the valve insert using a polymer material. E.g. the valve insert can be easily produced by injection molding or 3D printing/additive manufacturing. The present disclosure thus inter alia provides advantages with respect to manufacture, assembly and costs.

The first valve cylinder, the second valve cylinder and the flange may be formed, preferably molded, especially preferred injection-molded, integrally/in one piece/in one piece of material. The material of the valve insert is especially preferred a polymer/plastic material. The plurality of tube ports may also be formed in one piece/integrally with the first valve cylinder, the second valve cylinder and the flange. The valve insert/housing (comprising the first valve cylinder, the second valve cylinder, the flange and the plurality of tube ports) as a whole is preferably formed as a one piece/integral part/component and is thus preferably a single molded part having one combined body for at least two valves. As the valve insert is especially preferred an injection-molded part, it can be produced in high quantities with minimal costs.

The first valve cylinder may be a part of a first valve, and the second valve cylinder may be a part of a second valve. Preferably, the first valve is (functionally) separated from the second valve. Especially preferred, the first valve is provided for injection of gas (usually carbon dioxide) or water (gas/water injection valve), and the second valve is provided for suction (suction valve).

The first valve and the second valve may each comprise further components/parts like a piston or a button. There is however provided only one combined housing body (the valve insert) for both valves. The combined housing body (the valve insert) thus preferably forms the housing for/accommodates at least two valves, namely a suction valve and a gas/water injection valve. It is however to be understood that the valve insert of the present disclosure is not limited to accommodate exactly two valves. It may be provided that more than two valves, e.g. three or four valves, are accommodated in one single combined housing body (the valve insert).

Preferably, the first valve and the second valve are not fluidly interconnected, i.e. are functionally separate. This means that preferably two separate cylinders are provided and a first valve interior (surrounded by the first cylinder wall) is separated from a second valve interior (surrounded by the second cylinder wall). Said differently, there is preferably no functional intersection between the at least two valves.

The first valve (gas/water injection valve) preferably comprises a plurality of tube ports, e.g. at least four or even more. A tube port/tube ports of the gas/water injection valve may be connected to an air or water source/air or water sources, e.g. a water tank or an air pressure source. A tube port/tube ports of the gas/water injection valve may also be connected to tubes extending along the insertion cord towards the distal tip unit of the endoscope. The gas/water injection valve may be configured to be brought into different positions/states, in order to reach different purposes. E.g., the gas/water injection valve may be controlled such that a positive air pressure/gas source provides positive air pressure/gas (in particular carbon dioxide) to an insufflation tube provided in the insertion cord. In addition, the gas/water injection valve may also be controlled such that a water tank provides water under pressure to a rinsing tube provided in the insertion cord.

The second valve (suction valve) preferably has two tube ports, namely a first tube port connected to a working channel of the endoscope (preferably via a suction tube and a biopsy connector having a biopsy valve) and a second tube port connected to a suction device like a vacuum pump (via another suction tube). The suction valve is preferably configured to control a suction through the working channel, i.e. a suction that shall be applied to an object to be examined (e.g. hollow organ or body cavity). In particular, the suction valve may be brought into a valve closed state (in which no suction is applied to said object) and a valve opened state (in which suction is applied to said object).

The flange preferably extends radially from the first cylinder wall and from the second cylinder wall. I.e., the flange preferably has an extension direction, which is perpendicular to axes of both cylinders. That also means that the first valve cylinder is preferably oriented in parallel to the second valve cylinder.

The flange is preferably formed as a (thin-walled) plate-like flange. Said differently, the flange is preferably formed as a flat sheet/piece of a rigid/hard material, preferably of the same (rather small) thickness everywhere, limited on two opposite sides by a very extensive flat surface in relation to the thickness. Especially preferred, the plate-like flange has an angular/square shape, in particular a rectangular shape. It is to be understood that the plate-like flange may also have a shape different to an angular/square shape, e.g. a round shape so that the plate-like flange may be rather formed like a disc. It is only necessary that the plate-like flange is interrupted by both the first valve cylinder and the second valve cylinder so that the flange extends around and away from and connects the first valve cylinder and the second valve cylinder. The flange may also be described (when considered detached/separated/unconnected from the first valve cylinder and the second valve cylinder) as a plate-like flange having two round apertures/openings for the first valve cylinder and the second valve cylinder. It is to be understood however, that the valve insert is preferably an integral part and that the flange and the at least two valve cylinders are preferably integrally formed.

The aperture of the endoscope handle has preferably a shape that corresponds to a shape of the flange, and the flange is preferably arranged flush with the aperture/with a surface of the endoscope handle in which the aperture is formed. The flange preferably constitutes an extension of a (flat) surface of the endoscope handle when the valve insert is inserted/assembled into the endoscope handle. The flange may be fixed to the aperture. According to a preferred embodiment, the aperture of the endoscope handle has a rectangular shape and the flange has a rectangular shape.

The tube ports (which may also be designated as tube connections) are preferably formed as pipe sockets/protruding pipe portions extending (radially) away from the first cylinder wall and/or the second cylinder wall. Tube ports may protrude from the first cylinder wall of the first valve cylinder. Tube ports may also protrude from the second cylinder wall of the second valve cylinder. The tube ports may have a (hollow) cylindrical shape. Providing tube ports which are formed as pipe sockets has the advantage that tubes can be easily connected to the tube ports, e.g. by plugging in or by placing the tube over/around the tube port. This has also the effect that the valve insert can be easily quality tested outside of the endoscope, as tubes can be easily connected with and disconnected from the pipe socket type tube ports.

According to an advantageous embodiment, at least one tube port of the plurality of tube ports comprises a barbed push-in connection portion integrally formed, in particular molded, with the at least one tube port. All tube ports of the plurality of tube ports may comprise said barbed push-in connection portion. The barbed push-in connection portion enables an easy connection of a tube to the tube port and thus in an advantageous way provides a tube connection (portion). In particular, the barbs/barbed hooks provided at the barbed push-in connection portions enable that a tube can be simply pushed over the tube port and the barbs engage with and firmly hold the tube. Preferably, a form fit/positive (inter-)locking is provided between the barbed push-in connection portion and the tube. There is thus no need anymore to provide an adhesive connection between the tube and the tube port. The barbed push-in connection portion is preferably formed already in the injection-molding process in which the valve insert is formed. Therefore, there is no need to later on fix/attach the barbed push-in connection portion to the valve insert, which eases the assembly of the endoscope.

In addition or alternatively, it is preferred, when at least one tube port of the plurality of tube ports comprises an integrated sealing portion, in particular an integrated sealing O-ring. All tube ports of the plurality of tube ports may comprise said integrated sealing portion. Providing the tube port with an integrated sealing portion has the advantage, that no insertion of a separate sealing ring into the tube port is necessary later on, which also eases the assembly of the endoscope. Moreover, providing the integrated sealing portion may have the advantage that no inadvertent movement between the tube port and the separate sealing ring occurs during the pushing-in of the tube into the tube port. The integrated sealing portion is preferably formed already in the injection-molding process in which the valve insert is formed, so that there is no need to later on fix/attach the sealing portion to the valve insert. The integrated sealing portion is preferably adapted to the pipe socket type shape of the tube port and is thus preferably formed as a ring, in particular as a sealing O-ring.

Preferably, the integrated sealing portion is made of a second material, which is different from a first material from which a remainder of the valve insert is made. This makes it possible to choose a suitable (flexible) sealing material for the sealing portion, and to choose a suitable (rigid) material for the remainder of the valve insert.

It may be provided that at least one tube port of the plurality of tube ports comprises the/a barbed push-in connection portion and no integrated sealing portion, the barbed push-in connection portion being formed on an outer circumferential wall of the pipe socket type tube port, and configured such that a tube can be pushed over the tube port. The tube pushed over such a tube port is advantageously both fixed and sealed by the barbed push-in connection portion. Such a tube port is advantageous for rather large-diameter and flexible tubes. It is conceivable that all tube ports of the plurality of tube ports comprise the barbed push-in connection portion and no integrated sealing portion.

It may also be provided that at least one tube port of the plurality of tube ports comprises the/an integrated sealing portion and no barbed push-in connection portion, the integrated sealing portion being formed on an inner circumferential wall of the pipe-socket type tube port, and configured such that a tube can be pushed into the tube port. The tube pushed into such a tube port is advantageously both fixed and sealed by the integrated sealing portion. Such a tube port may be provided for rather small-diameter and rigid tubes. It is conceivable that all tube ports of the plurality of tube ports comprise the integrated sealing portion and no barbed push-in connection portion.

There may be also provided that the valve insert comprises at least one tube port having a/the barbed push-in connection portion and no integrated sealing portion and at least one tube port having an/the integrated sealing portion and no barbed push-in connection portion. There may thus be provided a tube port/tube ports having only the barbed push-in connection portion and a tube port/tube ports having only the integrated sealing portion on one single valve insert. In this case, a diameter of the tube port having the barbed push-in connection portion is especially preferred larger than a diameter of the tube port having the integrated sealing portion.

According to a further preferred embodiment it may also be provided that at least one tube port of the plurality of tube ports comprises: a barbed push-in connection portion integrally formed, in particular molded, with the at least one tube port; and an integrated sealing portion, in particular an integrated sealing O-ring, integrally formed, in particular molded, with the at least one tube port; wherein the barbed push-in connection portion is made of a first material, and the integrated sealing portion is made of a second material, wherein the first material is different from the second material. A remainder of the valve insert is particularly preferred made of the first material. According to said embodiment one tube port may thus comprise both a/the barbed push-in connection portion and an/the integrated sealing portion. Such a design may provide both a very good fixation and a very good sealing.

The valve insert may basically be a multi-component (in particular two-component) injection molded part and the first material may be a rigid polymer material and the second material may be a flexible polymer material. The first material may have a higher modulus of elasticity/elastic modulus than the second material. This means that the first material has preferably a higher stiffness/tensile strength than the second material. In other words, the first material is preferably more rigid than the second material or rather the second material is preferably more flexible than the first material.

Moreover, the first material and the second material may be both polymer materials. In this case, the valve insert having the first and the second material is cheap to manufacture and thus appropriate for a single use endoscope. Especially preferred, the first material is a rigid polymer material (e.g. acrylonitrile-butadiene-styrene (ABS), polycarbonate (PC), etc.) and the second material is a flexible polymer material (e.g. thermoplastic polyurethane (TPE), silicone, etc.). According to the present disclosure, “rigid” and “flexible” shall preferably be understood in relation to a use situation, when a user applies ordinary pressure by his fingers. In particular, a “flexible” material is preferably a material, which deforms in this use situation, and a “rigid” material is preferably a material, which does not deform in this use situation.

The (single-use) endoscope of the present disclosure is preferably a gastroscope, a colonoscope or a duodenoscope.

The present disclosure also relates to a system comprising an endoscope as described above and a monitor.

### BRIEF DESCRIPTION OF FIGURES

The disclosure is explained in more detail below using preferred embodiments and referring to the accompanying figures.

FIG. 1 is a plan view showing an endoscope according to the present disclosure;

FIG. 2 is a perspective view showing a portion of a housing of an endoscope handle and a valve insert according to a first embodiment in a non-assembled state;

FIG. 3 is a perspective view showing the portion of the housing of the endoscope handle and the valve insert according to the first embodiment in an assembled state;

FIG. 4 is a perspective view showing the valve insert according to the first embodiment from a rear side;

FIG. 5 is a perspective view showing a valve insert according to a second embodiment;

FIG. 6 is a perspective view showing a valve insert according to a third embodiment;

FIG. 7 is a cross-sectional view showing a valve cylinder of the valve insert according to the third embodiment; and

FIG. 8 is a top view showing a preferred variation of a tube port according to the disclosure.

The figures are schematic in nature and serve only to understand the disclosure. Identical elements are marked with the same reference signs. The features of the different embodiments can be interchanged among each other.

### DETAILED DESCRIPTION OF PREFERRED EMBODIMENTS

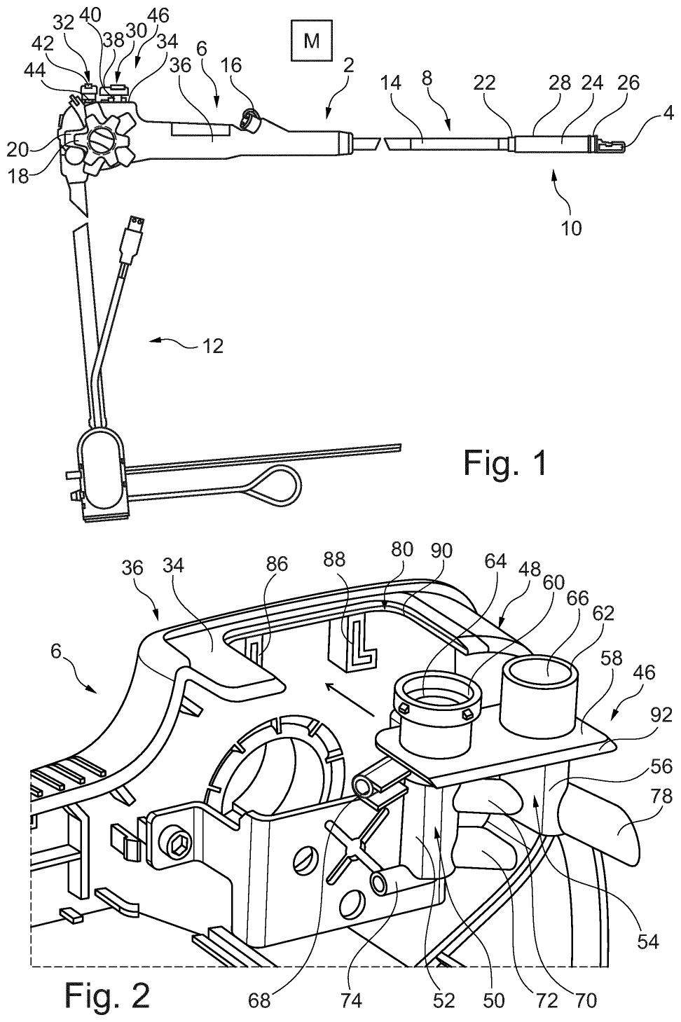

In FIG. 1, an endoscope 2 is shown. The endoscope 2 is preferably a single-use endoscope. The endoscope 2 comprises a distal tip unit 4, which is configured to be inserted into a patient's body cavity. Further, the endoscope 2 comprises a proximal endoscope handle 6 designed to be held by a user/physician and being configured to accommodate operating parts of the endoscope 2. Moreover, the endoscope 2 comprises an insertion tube 8 and a bending section 10, the insertion tube 8 and the bending section 10 extending from the endoscope handle 6 to the distal tip unit 4 and thus connecting the endoscope handle 6 and the distal tip unit 4. Said differently, the endoscope 2 comprises an endoscope handle 6 and an insertion cord, wherein the insertion cord comprises the insertion tube 8, the bending section 10 and the distal tip unit 4, which extend in this order from the endoscope handle 6. Moreover, a connector unit 12 for connecting the endoscope 2 to a supply unit is shown in FIG. 1.

At the distal tip unit 4, image capturing means such as a miniature video camera and illuminating means such as light-emitting diodes or optical fibers connected to a proximal source of light are arranged/installed, such that the patient's body cavity can be illuminated and inspected. An image captured by the image capturing means can be shown on a monitor M. The monitor M is provided separately from and connected with the endoscope 2.

The endoscope 2 has an internal working channel 14, which is formed as a bendable/flexible tube (not shown in FIG. 1). The working channel 14 is provided in/inside the insertion tube 8 and the bending section 10 and extends from the endoscope handle 6 towards the distal tip unit 4. The working channel 14 is accessible via an access port 16. In particular, a surgical instrument may be guided through the working channel 14 into the patient's body cavity via the access port 16. The user/physician is thus able to perform medical operations such as examinations within the patient's body cavity with the surgical instrument.

The endoscope handle 6 comprises two handle wheels, namely a first handle wheel 18 and a second handle wheel 20, for steering the distal tip unit 4. In particular, the first handle wheel 18 and the second handle wheel 20 can both be rotated/turned by the user. The first handle wheel 18 and the second handle wheel 20 are arranged coaxially, i.e. can be rotated around a common rotational axis.

The distal tip unit 4 may be tilted/bent/moved by bending the bending section 10 of the insertion cord. The endoscope 2 shown in FIG. 1 is basically a two-plane bending endoscope. This means that the distal tip unit 4 may bend in a first bending plane (e.g. in an up-and-down direction) and in a second bending plane (e.g. in a right-and-left direction). In particular, one of the handle wheels 18, 20, e.g. the first handle wheel 18, can be operated by the user to bend the distal tip unit 4 in the first bending plane and the other one of the handle wheels 18, 20, e.g. the second handle wheel 20, can be operated by the user to bend the distal tip unit 4 in the second bending plane. The first bending plane is preferably perpendicular to the second bending plane.

The bending section 10 comprises a plurality of segments including a proximal end segment 22, a plurality of intermediate segments 24 and a distal end segment 26. Two adjacent segments among the plurality of segments, i.e. a pair of segments, may be connected via corresponding flexible hinge members, respectively. The bending section 10 may be largely covered by a flexible cover 28 for preventing contamination.

The endoscope 2, in particular the endoscope handle 6, further comprises two valves, namely a gas/water injection valve 30 and a suction valve 32. The gas/water injection valve 30 and the suction valve 32 are arranged side by side on a top surface 34 of a housing 36 of the endoscope handle 6.

The gas/water injection valve 30 is preferably connected to both an gas/air source, in particular a gas cylinder/bottle (preferably filled with carbon dioxide), and a water source, in particular a water tank/bottle, via the connector unit 12. An insufflator may be provided to pump both gas and water into the endoscope 2. The gas/water injection valve 30 can be brought into different positions/states, in order to reach different purposes. In particular, the gas/water injection valve 30 can be controlled such that gas, in particular CO2, is provided to an insufflation tube provided in the insertion cord, or controlled such that water is provided to a rinsing tube provided in the insertion cord.

The suction valve 32 is preferably connected to the working channel 14 and—via the connector unit 12—to a suction device like a vacuum pump. The suction valve 32 can be brought into a valve closed state, in which no suction is applied to the working channel 14 and to a valve open state, in which suction is applied to the working channel 14.

As can be already seen in FIG. 1, the gas/water injection valve 30 comprises a first button 38 and a first piston 40, and the suction valve 32 comprises a second button 42 and a second piston 44. According to the present disclosure there is provided only one combined housing body that forms a housing for both valves, i.e. for both the gas/water injection valve 30 and the suction valve 32. This one combined housing body will be designated as valve insert 46 in the following.

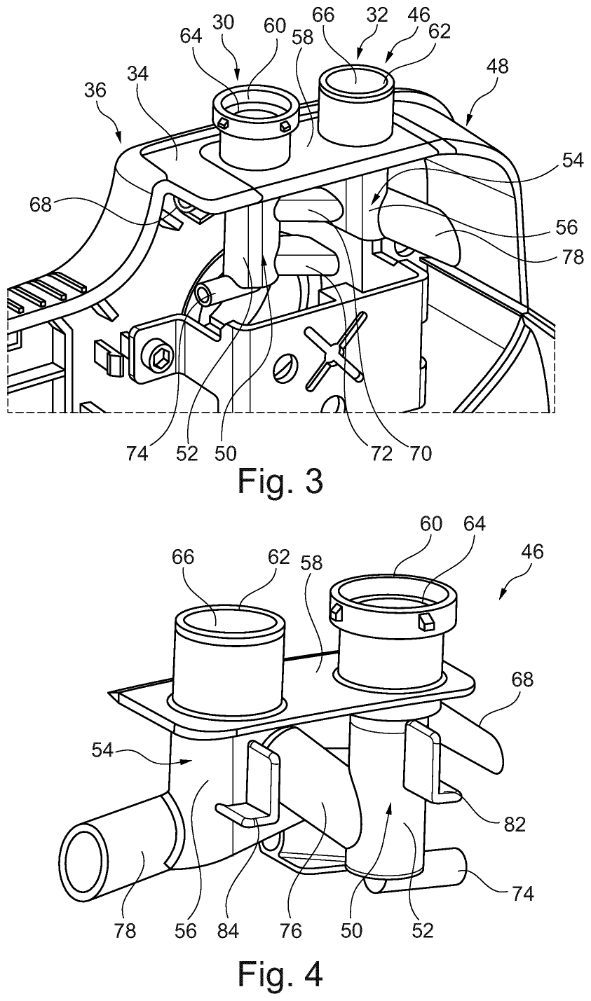

FIG. 2 is a perspective view showing a portion, in particular a half shell 48, of the housing 36 of the endoscope handle 6 and the valve insert 46 according to a first embodiment in a non-assembled state. FIG. 3 is a perspective view showing an assembled state of the valve insert 46 in the half shell 48. FIG. 4 is a perspective view of the valve insert 46 according to the first embodiment and shows the valve insert 46 from a rear side, whereas FIG. 2 and FIG. 3 show the valve insert 46 according to the first embodiment from a front side. The valve insert 46 according to the first embodiment will be described in the following with reference to FIG. 2, FIG. 3 and FIG. 4.

The valve insert 46 comprises a first valve cylinder 50 having a first cylinder wall 52, a second valve cylinder 54 having a second cylinder wall 56, and a flange 58 connecting and spacing apart the first valve cylinder 50 and the second valve cylinder 54.

The first valve cylinder 50 and the second valve cylinder 54 have a cylindrical shape and are both formed as hollow cylinders. The first valve cylinder 50 and the second valve cylinder 54 are oriented in parallel with respect to each other. The cylindrical shape of the first valve cylinder 50 is formed by the first cylinder wall 52, which has a constant, small wall thickness. The cylindrical shape of the second valve cylinder 54 is formed by the second cylinder wall 56, which also has a constant, small wall thickness.

The flange 58 is formed as a thin-walled, plate-like flange and has an essentially rectangular shape. The flange 58 extends radially from the first cylinder wall 52 and from the second cylinder wall 56 and has an extension direction which is perpendicular to axes of the first valve cylinder 50 and of the second valve cylinder 54.

The first valve cylinder 50 is a part of the gas/water injection valve 30. The second valve cylinder 54 is a part of the suction valve 32.

The first valve cylinder 50 has a top opening 60, which is visible and accessible from outside in an assembled state of the endoscope 2. I.e. the top opening 60 is positioned/arranged above/on an endoscope handle outer side with respect to the flange 58. Said differently, a portion of the first valve cylinder 50 is arranged above/on an endoscope handle outer side with respect to the flange 58 in an assembled state of the endoscope 2. The first button 38 and the first piston 40 are inserted into the top opening 60 in the assembled state of the gas/water injection valve 30.

Moreover, also the second valve cylinder 54 has a top opening 62, which is visible and accessible from outside in the assembled state of the endoscope 2. I.e. the top opening 62 is also arranged above/on an endoscope handle outer side with respect to the flange 58. Said differently, also a portion of the second valve cylinder 54 is arranged above/on an endoscope handle outer side with respect to the flange 58 in an assembled state of the endoscope 2. The second button 42 and the second piston 44 are inserted into the top opening 62 in the assembled state of the suction valve 32.

The gas/water injection valve 30 is functionally entirely separated from the suction valve 32 (no fluidic interconnection). I.e. the gas/water injection valve 30 and the suction valve 32 are only structurally connected to each other through the flange 58 of the valve insert 46. A first valve interior 64 surrounded by the first cylinder wall 52 is thus entirely separated from a second valve interior 66 surrounded by the second cylinder wall 56.

The gas/water injection valve 30/the first valve cylinder 50 comprises four tube ports, namely a first tube port 68, a second tube port 70, a third tube port 72 and a fourth tube port 74. The suction valve 32/the second valve cylinder 54 comprises two tube ports, namely a fifth tube port 76 and a sixth tube port 78. The tube ports 68, 70, 72, 74, 76 and 78 are all formed as pipe sockets/protruding pipe portions and have a hollow cylindrical shape. The tube ports 68, 70, 72 and 74 extend radially away from the first cylinder wall 52 of the first valve cylinder 50. The tube ports 76 and 78 extend radially away from the second cylinder wall 56 of the second valve cylinder 54. An extension direction of the tube ports 68, 70, 72, 74, 76 and 78 is preferably either a first, distal direction or a second, proximal direction, which is opposite to the first, distal direction (due to limited assembly space). All tube ports 68, 70, 72, 74, 76 and 78 are arranged at an endoscope inner side with respect to the flange 58.

The first tube port 68 of the first valve cylinder 50 is arranged close to/adjacent the flange 58 on an endoscope inner side with respect to the flange 58 and extends distally/opposite a direction to the second valve cylinder 54, i.e. away from the second valve cylinder 54. The first tube port 68 may be connected to a rinsing tube provided in the insertion cord.

The second tube port 70 of the of the first valve cylinder 50 is arranged close to/adjacent the flange 58 on an endoscope inner side with respect to the flange 58 and extends proximally/in a direction towards the second valve cylinder 54. The second tube port 70 may be connected to a water tank/bottle.

The third tube port 72 of the first valve cylinder 50 is arranged away from the flange 58/near an end of the first valve cylinder 50 on an endoscope inner side with respect to the flange 58 and extends proximally/in a direction towards the second valve cylinder 54. The third tube port 72 may be connected to a gas bottle.

The fourth tube port 74 of the first valve cylinder 50 is arranged away from the flange 58/at an end of the first valve cylinder 50 on an endoscope inner side with respect to the flange 58 and extends distally/opposite a direction to the second valve cylinder 54. The fourth tube port 74 may be connected to an insufflation tube provided in the insertion cord.

The gas/water injection valve 30 can be brought into different positions/states, in order to reach different purposes. E.g. water can be provided from the water tank/bottle to the second tube port 70 and leave the gas/water injection valve 30 via the first tube port 68 towards the rinsing tube, or air/gas can be provided from the gas bottle to the third tube port 72 and leave the gas/water injection valve 30 via the fourth tube port 74 towards the insufflation tube.

The fifth tube port 76 of the second valve cylinder 54 is arranged close to/adjacent the flange 58 on an endoscope inner side with respect to the flange 58 and extends distally/in a direction towards the first valve cylinder 50. The fifth tube port 76 of the second valve cylinder 54 may be connected to the working channel 14.

The sixth tube port 78 of the second valve cylinder 54 is arranged away from the flange 58/near an end of the second valve cylinder 54 on an endoscope inner side with respect to the flange 58 and extends proximally/in a direction away from the first valve cylinder 50. The sixth tube port 78 of the suction valve 32 may be connected to a suction device like a vacuum pump.

The suction valve 32 can be brought into a valve open state in which suction is applied to the working channel 14, and into a valve closed state in which no suction is applied to the working channel 14.

The first valve cylinder 50, the second valve cylinder 54, the flange 58 and the tube ports 68, 70, 72, 74, 76 and 78 are formed/molded integrally in one piece of polymer material and in their entirety form the valve insert 46.

As can be seen from FIG. 2 and FIG. 3, the top surface 34 of the housing 36 of the endoscope handle 6, in particular of the half shell 48, comprises an aperture/opening 80. The aperture 80 has basically a rectangular shape (optionally with rounded corners). The flange 58 of the valve insert 46 can be arranged in the aperture 80.

The valve insert 46 is connected to the half shell 48 of the housing 36 by inserting preferably L-shaped alignment protrusions 82, 84 provided at and radially extending away from the first cylinder wall 52 of the first valve cylinder 50 and from the second cylinder wall 56 of the second valve cylinder 54 into corresponding preferably L-shaped alignment recesses 86, 88 provided on an inner side of the half shell 48 of the housing 36. Moreover, the aperture 80 has a step-like formation and comprises a step 90 on which the flange 58 is supported in the assembled state of the endoscope 2. The flange 58 preferably also has a step-like formation comprising a step 92, which is provided for easy assembling of another (second) half shell of the housing 36 to the half shell 48 and the valve insert 46. As it can be seen from FIG. 3, the flange 58 is arranged flush with the top surface 34 of the housing 36 when arranged in the aperture 80 and thus constitutes an extension of the flat top surface 34.

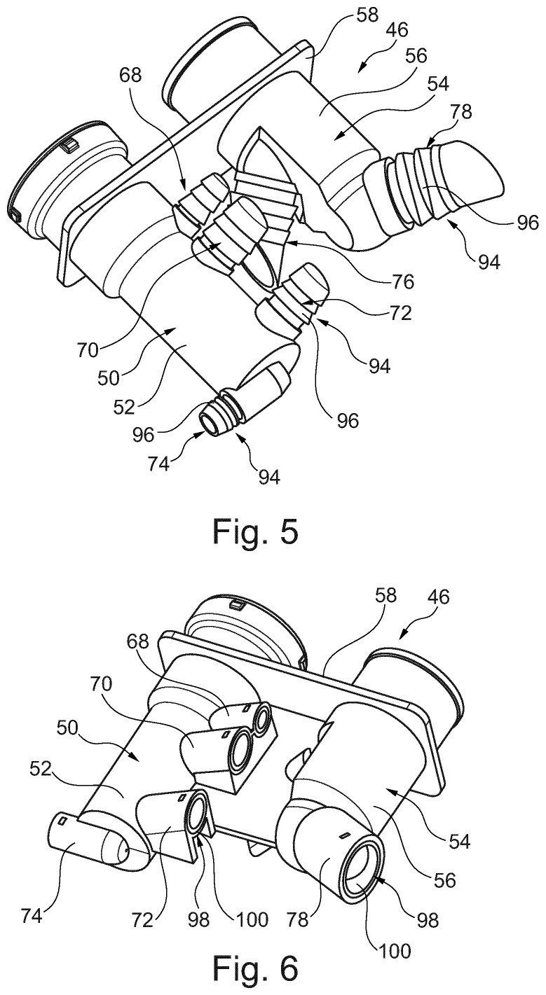

FIG. 5 is a perspective view of a valve insert 46 according to a second embodiment. The second embodiment differs from the first embodiment first of all in the arrangement/orientation of the first tube port 68 of the first valve cylinder 50. As it can be seen from FIG. 5, the first tube port 68 extends in a proximal direction (and not in the distal direction). Apart from that it can be seen that all tube ports 68, 70, 72, 74, 76 and 78 comprise a barbed push-in connection portion 94 having barbs 96. The barbs 96 can engage and firmly hold and seal a tube pushed over the respective tube port 68, 70, 72, 74, 76 and 78. The barbs 96 forming the barbed push-in connection portions 94 are preferably already formed in the injection-molding process of the valve insert 46. Apart from that the description of the first embodiment applies mutatis mutandis for the second embodiment and is not repeated.

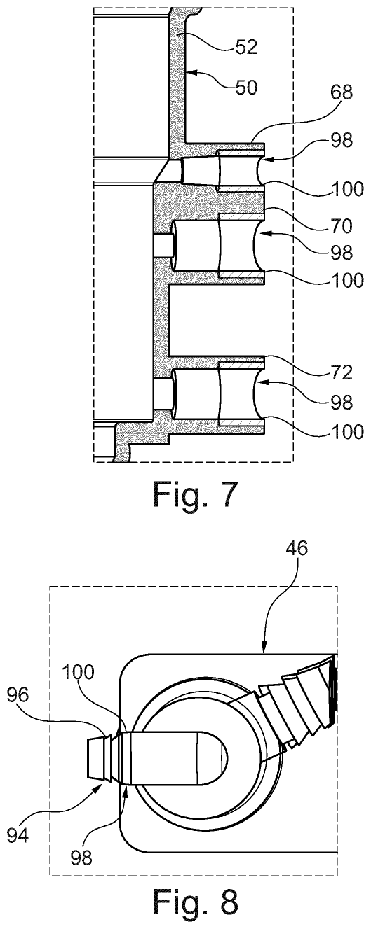

FIG. 6 is a perspective view of a valve insert 46 according to a third embodiment. FIG. 7 shows a cross-sectional view of the first valve cylinder 50 (being a part of the gas/water injection valve 30) of the valve insert 46 according to the third embodiment. The third embodiment differs from the first embodiment also in the orientation of the first tube port 68 extending in a proximal direction (and not in the distal direction). Apart from that it can be seen that all tube ports 68, 70, 72, 74, 76 and 78 comprise an integrated sealing portion 98 formed as an integrated sealing O-ring 100. The integrated sealing portion 98/the sealing O-ring 100 is preferably formed already in the injection-molding process in which the valve insert is formed. The integrated sealing portion 98/the sealing O-ring 100 is formed from a flexible sealing material like thermoplastic polyurethane or silicone, whereas a remainder of the valve insert 46 is formed from a rigid material like acrylonitrile-butadiene-styrene (ABS) or polycarbonate (PC). The valve insert 46 is preferably injection-molded in a two-component injection molding process. The integrated sealing portion 98/the sealing O-ring 100 is preferably formed on an inner wall surface of the respective tube port 68, 70, 72, 74, 76, 78. Apart from that the description of the first embodiment applies mutatis mutandis for the third embodiment and is not repeated.

It is to be understood that—although not shown in the first embodiment—also the tube ports 68, 70, 72, 74, 76 and 78 of the valve insert 46 of the first embodiment shown in FIG. 2, FIG. 3 or FIG. 4 may comprise the barbed push-in connection portion 94 having the barbs 96 and/or the integrated sealing portion 98 formed as the sealing O-ring 100. E.g. it is advantageous to provide tube ports having a rather small diameter with the integrated sealing portion 98, and to provide tube ports having a rather large diameter with the barbed push-in connection portion 94. In this light, e.g. the first tube port 68 and the fourth tube port 74 may be provided with the integrated sealing portion 98, and the remaining tube ports 70, 72, 76 and 78 may be provided with the barbed push-in connection portion 94 in the first embodiment. The reason for that is that small diameter tubes are rather rigid and are appropriately sealed and held by the integrated sealing portions 98, whereas large diameter tubes are rather flexible and are appropriately sealed and held by the barbed push-in connection portion 94.

As can be seen in FIG. 8, according to a variation of the present disclosure relating to all embodiments, it may also be provided that one tube port, several tube ports or all tube ports of the plurality of tube ports 68, 70, 72, 74, 76 and 78 comprise(s) both the barbed push-in connection portion 94 and the integrated sealing portion 98. E.g., as can be seen in FIG. 8, it may be provided that both the barbed push-in connection portion 94 and the integrated sealing portion 98 are formed on an outer wall surface of the respective tube port 68, 70, 72, 74, 76, 78.

### LIST OF REFERENCE SIGNS

2 endoscope
4 distal tip unit
6 endoscope handle
8 insertion tube
10 bending section
12 connector unit
14 working channel
16 access port
18 first handle wheel
20 second handle wheel
22 proximal end segment
24 intermediate segment
26 distal end segment
28 cover
30 gas/water injection valve or first valve
32 suction valve or second valve
34 top surface
36 housing (of endoscope handle)
38 first button (of gas/water injection valve)
40 first piston (of gas/water injection valve)
42 second button (of suction valve)
44 second piston (of suction valve)
46 valve insert
48 half shell
50 first valve cylinder
52 first cylinder wall
54 second valve cylinder
56 second cylinder wall
58 flange
60 top opening (of first valve cylinder)
62 top opening (of second valve cylinder)
64 first valve interior
66 second valve interior
68 first tube port
70 second tube port
72 third tube port
74 fourth tube port
76 fifth tube port
78 sixth tube port
80 aperture
82 first alignment protrusion
84 second alignment protrusion
86 first alignment recess
88 second alignment recess
90 step (of aperture)
92 step (of flange)
94 barbed push-in connection portion
96 barb
98 integrated sealing portion
100 O-ring
M monitor

1-15. (canceled)

16. An endoscope comprising:
a handle having an aperture; and
a valve insert arranged in the aperture, the valve insert comprising:
a first valve cylinder having a first cylinder wall,
a second valve cylinder having a second cylinder wall, and
a flange connecting and spacing apart the first valve cylinder and the second valve cylinder, the flange being arranged in the aperture of the endoscope handle, wherein a portion of the first valve cylinder and a portion of the second valve cylinder are arranged inside the handle and comprise tube ports.

17. The endoscope of claim 16, wherein the first valve cylinder, the second valve cylinder, the flange, and the tube ports are formed in one piece, wherein the flange extends radially from the first cylinder wall and from the second cylinder wall, wherein the handle comprises a housing wall, the aperture is formed in the housing wall, the aperture has a shape that corresponds to a shape of the flange, and the flange is arranged flush with the housing wall.

18. The endoscope of claim 16, wherein the first valve cylinder, the second valve cylinder and the flange are formed in one piece.

19. The endoscope of claim 18, wherein the tube ports are formed in one piece with the first valve cylinder, the second valve cylinder and the flange.

20. The endoscope of claim 18, wherein the first valve cylinder is a part of a first valve, the second valve cylinder is a part of a second valve, the first valve is functionally separated from the second valve, the first valve is configured to inject gas or water, and the second valve is configured for suction.

21. The endoscope of claim 18, wherein the flange extends radially from the first cylinder wall and from the second cylinder wall.

22. The endoscope of claim 21, wherein the flange is formed as a plate-like flange.

23. The endoscope of claim 22, wherein the aperture has a shape that corresponds to a shape of the flange, and the flange is arranged flush with the aperture.

24. The endoscope of claim 16, wherein the tube ports are formed as pipe sockets extending away from the first cylinder wall and/or the second cylinder wall.

25. The endoscope of claim 16, wherein at least one tube port of the tube ports comprises a barbed push-in connection portion integrally formed with the at least one tube port.

26. The endoscope of claim 16, wherein at least one tube port of the tube ports comprises an integrated sealing portion.

27. The endoscope of claim 26, wherein the integrated sealing portion comprises a sealing O-ring.

28. The endoscope of claim 26, wherein the integrated sealing portion is made of a second material, which is different from a first material from which the first valve cylinder, the second valve cylinder, and the flange are made.

29. The endoscope of claim 16, wherein at least one tube port of the tube ports comprises:
a barbed push-in connection portion integrally formed with the at least one tube port; and
an integrated sealing portion integrally formed with the at least one tube port,
wherein the barbed push-in connection portion is made of a first material,
wherein the first cylinder wall, the second cylinder wall, and the flange are made of a second material, and
wherein the first material is different from the second material.

30. The endoscope of claim 16, wherein the valve insert is a multi-component injection molded part comprising a rigid polymer material and a flexible polymer material.

31. The endoscope of claim 30, wherein the first cylinder wall, the second cylinder wall, and the flange are made of the rigid material.

32. The endoscope of claim 31, further comprising barbed push-in connection portions and/or integrated sealing portions formed of the flexible material integrally with at least some of the tube ports.

33. The endoscope of claim 16, wherein at least some of the tube ports comprise barbed push-in connection portions integrally formed with the at least some of the tube ports, the barbed push-in connection portions made of a material different from a material from which the first cylinder wall, the second cylinder wall, and the flange are made.

34. The endoscope of claim 33, further each of at least some of the tube ports comprises an integrated sealing portion.

35. A system comprising:
the endoscope of claim 16; and
a monitor.

36. An endoscope comprising:
a handle having an aperture; and
a valve insert arranged in the aperture, the valve insert comprising:
a first valve cylinder having a first cylinder wall,
a second valve cylinder having a second cylinder wall, and
a flange connecting and spacing apart the first valve cylinder and the second valve cylinder, the flange being arranged in the aperture of the endoscope handle,
wherein a portion of the first valve cylinder and a portion of the second valve cylinder are arranged inside the handle and comprise tube ports,
wherein the first valve cylinder, the second valve cylinder, the flange, and the tube ports are formed in one piece of a first material,
wherein the flange extends radially from the first cylinder wall and from the second cylinder wall,
wherein the handle comprises a housing wall, the aperture is formed in the housing wall, and the aperture has a shape that corresponds to a shape of the flange,
wherein the tube ports comprise a first tube port and a second tube port, the first tube port being larger in cross-section than the second tube port, the first tube port comprising an integrally formed barbed push-in connection portion, and the second tube port comprising an integrated sealing portion.

37. The endoscope of claim 36, wherein the handle comprises a housing wall having an outer surface, the aperture is formed in the housing wall, the aperture has a shape that corresponds to a shape of the flange, and the flange is arranged flush with the outer surface of the housing wall.

38. A system comprising:
the endoscope of claim 36; and
a monitor.
